(19) United States
(12) Patent Application Publication
(10) Pub. No.: US 2021/0282669 A1
(43) Pub. Date: Sep. 16, 2021

(54) SYSTEM AND METHOD FOR MONITORING EYE BLINK MECHANICS

(71) Applicants: Peter Borden, San Mateo, CA (US); Harvey Fishman, Menlo Park, CA (US)

(72) Inventors: Peter Borden, San Mateo, CA (US); Harvey Fishman, Menlo Park, CA (US)

(21) Appl. No.: 16/819,059

(22) Filed: Mar. 14, 2020

(51) Int. Cl. A61B 5/11; A61B 3/113; A61B 3/10; A61B 5/00

(52) U.S. Cl. CPC A61B 5/1103; A61B 3/113; A61B 3/101; A61B 5/6803; A61B 2562/02; A61B 5/7405; A61B 5/742; A61B 5/4836; A61B 5/0022

(57) ABSTRACT

A system and method for monitoring eye blink mechanics is disclosed herein. One or more sensors mounted on an eyeglass frame may record blink mechanics over an extended period of time, typically longer than can be observed by a doctor during the course of a clinical examination, using camera imaging or infrared reflection. The system and method for monitoring eye blink mechanics provides a simultaneous record of blink rate and drying of the tear film, and may also monitor completeness of the individual blink process. Recorded data may be downloaded to a data processing system for analysis, the results of which are used to indicate treatment for DES, DBM, and other blink related pathologies. The system may also provide treatment through feedback based on real-time measurements by training the patient to correct dysfunctional blink mechanics as they are occurring.

### TECHNICAL FIELD OF THE INVENTION

The present invention relates in general to ophthalmic devices, and, more specifically, to a system and method for monitoring eye blink mechanics.

### COPYRIGHT AND TRADEMARK NOTICE

A portion of the disclosure of this patent application may contain material that is subject to copyright protection. The owner has no objection to the facsimile reproduction by anyone of the patent document or the patent disclosure, as it appears in the Patent and Trademark Office patent file or records, but otherwise reserves all copyrights whatsoever.

Certain marks referenced herein may be common law or registered trademarks of third parties affiliated or unaffiliated with the applicant or the assignee. Use of these marks is by way of example and should not be construed as descriptive or to limit the scope of this invention to material associated only with such marks.

### BACKGROUND OF THE INVENTION

Dry Eye Syndrome (DES) is one of the most common sources of eye pain. In many cases this pain results from the tear film evaporating between blinks and causing drying of the sensitive corneal and scleral surfaces. This pain can happen when the blink frequency is too low, tear production is insufficient, or a combination of the two.

The present patent disclosure addresses one of the best known, though under-appreciated, etiologies of DES: Dysfunctional Blink Mechanics (DBM). The present disclosure relates to a device to quantify and diagnose DBM, and methods to use the device to treat DBM and train the patient to address DBM.

There are three interrelated factors of blink mechanics that affect DES: tear film integrity, blink rate, and completeness of the blink.

By way of example, a person with insufficient tear production may need a higher blink rate to keep the eye surface wet, and a person with a high blink rate may still feel irritation if the blinks are incomplete. Incomplete blinking also allows residue to build up in the tear fluid at the base of the eye opening, thus exacerbating irritation.

In the same way that a Holter monitor tracks heart activity during daily activity, it would be desirable to monitor blink mechanics during daily activity. Such a capability is not available in modern clinical practice.

Beyond a quick look during a clinical visit there is no means to monitor for extended periods, and examination of blink mechanics is not part of the standard of modern clinical practice. Blink mechanics are, in fact, not easily observed in the clinical setting as they depend on daily activity. For example, walking outside can cause the tear film to dry more rapidly with exposure to dry air or wind. Watching a computer screen can slow blink rate. In addition, blinks occur rapidly so the blink does not obstruct vision, which makes events such as incomplete blinking difficult to observe.

In a clinical setting it would be especially important to simultaneously monitor all three factors listed above. Monitoring blink rate alone is meaningless without knowledge of tear film integrity, and incomplete blinks at a high rate can still result in eye irritation.

Dysfunctional blink mechanics can be a significant problem for those with an inhibited blink reflex, such as persons with traumatic brain injury (TBI) or Parkinsonism. Even otherwise normal individuals may only partially blink and experience irritation in the lower portion of the eye.

Beyond discovery of issues related to blink mechanics, it is also desirable to train patients to improve their blink mechanics in real time, to directly address this fundamental cause of corneal discomfort and DES.

Thus, there is a need in the art for a system and method for monitoring eye blink mechanics that records blink activity over an extended period and during normal daily activity, and to use these measurements and the data obtained to treat DES, DBM, and other blink related pathologies. It is to these ends that the present invention has been developed.

### BRIEF SUMMARY OF THE INVENTION

To minimize the limitations in the prior art, and to minimize other limitations that will be apparent upon reading and understanding the present specification, the present invention describes a system and method for monitoring eye blink mechanics.

It is an objective of the present invention to provide a system and method for monitoring eye blink mechanics that may comprise a portable device.

It is another objective of the present invention to provide a system and method for monitoring eye blink mechanics that may comprise a wearable device.

It is another objective of the present invention to provide a system and method for monitoring eye blink mechanics that may be attached to eyeglasses.

It is another objective of the present invention to provide a system and method for monitoring eye blink mechanics that may comprise a plurality of sensors.

It is another objective of the present invention to provide a system and method for monitoring eye blink mechanics that may comprise a plurality of inputs.

It is another objective of the present invention to provide a system and method for monitoring eye blink mechanics that may comprise a wireless connectivity.

It is another objective of the present invention to provide a system and method for monitoring eye blink mechanics that may comprise an audio output.

It is another objective of the present invention to provide a system and method for monitoring eye blink mechanics that may comprise a visual output.

It is another objective of the present invention to provide a system and method for monitoring eye blink mechanics that may comprise a single-component construction.

It is another objective of the present invention to provide a system and method for monitoring eye blink mechanics that may comprise a multiple-component construction.

It is another objective of the present invention to provide a system and method for monitoring eye blink mechanics that may comprise a resilient material of construction.

It is another objective of the present invention to provide a system and method for monitoring eye blink mechanics that may comprise a cleanable material of construction.

It is another objective of the present invention to provide a system and method for monitoring eye blink mechanics that may comprise a reusable material of construction.

It is another objective of the present invention to provide a system and method for monitoring eye blink mechanics that may comprise an antimicrobial layer.

It is another objective of the present invention to provide a system and method for monitoring eye blink mechanics that may comprise an antimicrobial material of construction.

It is another objective of the present invention to provide a system and method for monitoring eye blink mechanics that may measure blink rate.

It is another objective of the present invention to provide a system and method for monitoring eye blink mechanics that may measure tear film integrity.

It is another objective of the present invention to provide a system and method for monitoring eye blink mechanics that may measure blink completeness.

It is another objective of the present invention to provide a system and method for monitoring eye blink mechanics that may measure blink velocity.

It is another objective of the present invention to provide a system and method for monitoring eye blink mechanics that may measure parameters in a clinical setting.

It is another objective of the present invention to provide a system and method for monitoring eye blink mechanics that may measure parameters outside of a clinical setting.

These and other advantages and features of the present invention are described herein with specificity so as to make the present invention understandable to one of ordinary skill in the art, both with respect to how to practice the present invention and how to make the present invention.

### BRIEF DESCRIPTION OF THE SEVERAL VIEWS OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention.

FIG. 1 illustrates a plurality of components of a system and method for monitoring eye blink mechanics, as contemplated by the present disclosure;

FIG. 2 illustrates a principle of operation of a system and method for monitoring eye blink mechanics, as contemplated by the present disclosure;

FIG. 3 illustrates an example of waveforms as a function of time of a system and method for monitoring eye blink mechanics, as contemplated by the present disclosure;

FIG. 4 illustrates a blink monitor mounted on an exemplary glasses of a system and method for monitoring eye blink mechanics, as contemplated by the present disclosure;

FIG. 5 illustrates an exemplary circuit diagram of a system and method for monitoring eye blink mechanics, as contemplated by the present disclosure;

FIG. 6 illustrates an oscilloscope tracing of decaying blinks and decaying of tear film signal of a system and method for monitoring eye blink mechanics, as contemplated by the present disclosure;

FIG. 7 illustrates a recorded blink data stream of a system and method for monitoring eye blink mechanics, as contemplated by the present disclosure; and

FIG. 8 illustrates a plurality of blink readings dependent on multiple variables of a system and method for monitoring eye blink mechanics, as contemplated by the present disclosure.

### DETAILED DESCRIPTION OF THE INVENTION

Certain terminology is used in the following description for reference only and is not limiting. The words “front,” “rear,” “anterior,” “posterior,” “lateral,” “medial,” “upper,” “lower,” “outer,” “inner,” and “interior” refer to directions toward and away from, respectively, the geometric center of the invention, and designated parts thereof, in accordance with the present disclosure. Unless specifically set forth herein, the terms “a,” “an,” and “the” are not limited to one element, but instead should be read as meaning “at least one.” The terminology includes the words noted above, derivatives thereof, and words of similar import.

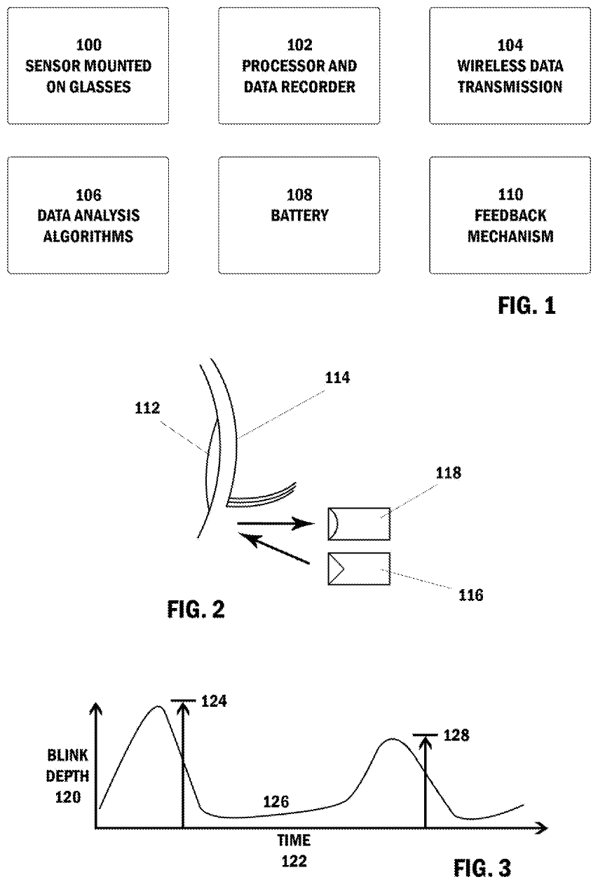

The illustrations of FIGS. 1-8 illustrate a system and method for monitoring eye blink mechanics, as contemplated by the present disclosure. The purpose of the system and method for monitoring eye blink mechanics is to provide a system and method that records blink activity over an extended period and during normal daily activity, and to use those measurements and the data obtained to treat Dry Eye Syndrome (DES), Dysfunctional Blink Mechanics (DBM), and other blink related pathologies.

The illustrations of FIGS. 1-8 illustrate a system and method for monitoring eye blink mechanics, as contemplated by the present disclosure. This invention describes a system to monitor blink mechanics over time, and methods to use the system to treat dysfunctional blinking and related eye disorders. It is portable, thus enabling it to take measurements over extended periods, for example, in a waiting room prior to an exam, or outside the clinic while the subject conducts normal activities. Blink mechanics can include, but are not restricted to, blink rate, the rate at which the eyelid closes and opens, whether the closure is full or incomplete, and drying of the eye surface between blinks. An additional factor is the velocity profile during the blink, which can relate to blink completeness and indicate other issues related to, for example, weakness or neurological conditions. These factors can be significant in diagnosing causes of DES and other blink-related disorders and, if corrected, can lead to significant reduction in the severity of such disorders.

Unique features of this device include the ability to monitor multiple related parameters concurrently, specifically blink rate, tear film integrity, and, in some embodiments, blink completeness (whether the eyelid fully or only partially closes) and velocity profile of the eyelid during a blink (blink velocity), and to do so while the patient conducts normal activities. This is significant because the purpose of blinking is to maintain an integral tear film and to flush out residue accumulating at the lower portion of the eye using complete blinks. Thus, tear film integrity determines the proper blink rate, and incomplete blinking will not adequately maintain a clean, integral tear film in the lower portion of the eye. Further, as the tear film integrity is a function of environmental conditions, it is essential to monitor blink dynamics and tear film integrity while conducting normal daily activities.

The device may be dispensed to a patient as part of an exam or treatment for a condition such as DES. Some embodiments may also be sold to consumers as a commercial device for self-monitoring and treatment.

The device consists of up to six components, indicated in FIG. 1. One or more sensors 100 mounted near the eye on a pair of eyeglasses, such as on the frame or on one or both lenses. Note that the lenses may be neutral or absent when the patient does not need correction. In one embodiment, the device clips on a frame worn on the head, such as the frame of a pair of eyeglasses or a pair of eyeglasses that may include lenses. Sensors 100 may use imaging with a camera, light reflectance, or a combination of the two. Some embodiments may use one or more sensors on one or both eyes, and may record one or multiple locations, such as the left and right side of the lower portion of an eye.

A processor and data recorder 102. This provides pre-processing of the signals to, for example, reduce the size of the data set or to improve signal-to-noise ratio. For example, the processor might dynamically adjust the signal baseline to correct for shifts in the signal should the eyeglasses change position, or pre-process an image to obtain real-time blink data. A memory to store data is also included. In one embodiment, this is a solid-state memory, such as an SD flash memory card, that can be removed from the device and read by a separate computer. In another embodiment this component is separate from the eyeglasses, connected either by a wire or a wireless connection such as Bluetooth to a remote device such as a smart phone. In one embodiment this component consists of an app running on a handheld device such as a smart phone. In another embodiment signal conditioning electronics mounts on an arm of the eyeglasses and further signal processing and data storage occurs on the remote device.

An optional wireless data transmission means 104 provides connection between various components of the system. In one embodiment, this includes a wireless, such as Bluetooth connection between the eyeglasses and a smart phone, which can, for example, act as a remote recording device that the patient carries while the device is collecting data. In another embodiment, this additionally includes a Wi-Fi or phone connection between a smart phone and a computer in a clinician's office, so that data collected can be sent to a clinician electronically.

A data analysis system 106 running one or more algorithms for analyzing and presenting the data to a user. This may run on a computer, a smart phone or a combination of the two. It takes the collected data, pre-processes it to, for example, remove noise, adjust the signal baseline or provide data compression to reduce file size, analyzes it for desired elements of blink mechanics, and provides a means to read out the results. For example, it may identify a series of peaks in the signal and compute the average time between peaks to determine the blink rate. In another example, it may find peaks of lower amplitude and identify them as incomplete or partial blinks. In another example, it may observe a change in the baseline between blinks and compute the time for the tear film to dry (sometimes called tear breakup time, or TBUT). In another example, it uses the rate of change of the blink signal to infer completeness of closure. Readouts may be in many forms, such as graphs, tables, and summary data. Note that this system may span multiple processors. In one embodiment, data is sent to a smart phone, analyzed locally, and sent electronically via, for example, a phone or internet connection, to a clinician's computer for further analysis and presentation as a report.

One or more batteries 108 to provide power for the sensor and processor/data recorder. In one embodiment, this is a lithium battery mounted on the eyeglass frame. In another embodiment, the battery is remote from the eyeglass frame and the power connection is through a cable.

Some embodiments include a feedback mechanism 110 to provide real-time feedback to the patient, indicating, for example, when the blink rate is too slow or blinking is incomplete, or to train the patient to improve blink mechanics. In one embodiment the system measures a parameter such as drying and tells the patient to increase blink rate if the tear film dries between blinks. In another embodiment the system measures blink completeness or rate of eye lid closure and opening and tells the patient to blink completely. In another embodiment the system determines when the tear film is drying too fast and indicates when to apply drops such as artificial tears. The feedback can be a signal such as an LED flash. In another embodiment, the signal is a beep. For example, a small beeper is located at the end of one of the arms of the eyeglasses, near the ear, so that beeps can be heard. The indications can be encoded. For example, a flash or beep can repeat at a rate equal to the desired blink rate. In another example, a double beep is used to tell the patient to blink stronger and a single beep tells the patient when to blink. In another embodiment, the processor 102 on the glasses includes a voice synthesizer that provides the patient explicit instructions such as when to blink or when to add eye drops. The advantage of this method is that it employs active feedback, so it trains the patient how to improve blink mechanics based upon measured blink parameters. Alternately, it provides treatment for patients with Parkinson's Disease or a similar syndrome where the blink reflex is otherwise suppressed. In another embodiment, the feedback signal is obtained from a smart device such as a handheld phone or watch.

In one embodiment LED or laser source 116 is mounted on the eyeglass frame 130 at the bridge and infrared detector 118 is mounted opposite source 116, also on the frame. Note that in other embodiments source 116 and detector 118 may be mounted at different sites, or may be adjacent to each other and mounted on the lens. An infrared beam from source 116 reflects off the sclera 112 to detector 118, at a site on sclera 112 below the open eyelid 114. The source 116 and detector 118 connect with small wires to housing 100 mounted on the frame 130 that contains a battery, signal conditioning electronics, and a wireless transmitter to send signals to a receiver such as a smart phone, which is not shown. The electronics in housing 100 may also connect to an LED light or sound source to provide feedback to the user on, for example, when to blink. In another embodiment, the infrared beam from source 116 reflects off the cornea.

The infrared beam comes from source 116, reflects off the sclera 112, and passes to the receiver 118. Source 116 and receiver 118 are mounted on frame 130.

The shape of the resulting signals 120 may be analyzed to find blink dynamics parameters. For example, a short, sharp signal is seen with an incomplete blink 128. A complete blink has a wider bottom because the eye lid 114 remains in the closed position a short time while it reverses direction. A still longer blink 124 indicates an intentional closure of the eye. The shape of the recovery 126 of the signal indicates tear drying. The signal, rather than immediately recovering, gradually recovers and rounds at the peak.

In one embodiment, the sensor is a small camera mounted on the eyeglass frame positioned to view the lower portion of an eye. It records eye closures over a period of time, which may be minutes, hours or days. In one embodiment, the video is processed so that only summary data is stored, rather than full video. This includes derived quantities such as the time a blink is recorded, whether the blink was full, or the rate of eye closing and opening. The video may be processed either in real time or post processed to extract information such as the speed of the eye lid as it closes and opens, the completeness of a blink, the time between blinks (or blink rate), and the extent of drying. In one embodiment a monochromatic light source, such as a laser diode or LED, illuminates the eye in order to show changes in reflection from the eye surface or interference fringes that indicate drying. This source is preferably in the infrared, with a wavelength longer than 640 nm, so that it is not visible, and so that risk of causing eye damage is minimal.

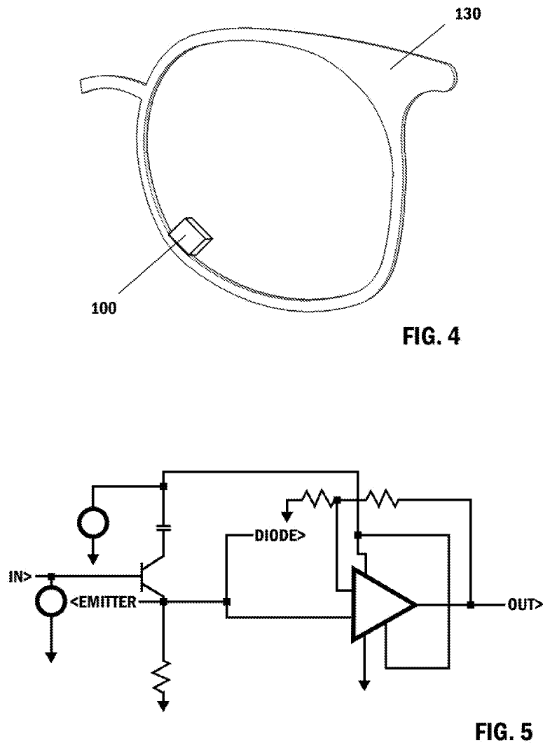

In another embodiment the sensor is a small light source and detector used to illuminate the eye and capture light reflected from the eye surface. This is described with the aid of FIG. 2. In one embodiment, an infrared source 116 (wavelength >640 nm) such as an LED or laser and infrared detector mount on a circuit board 100 that is affixed to the bottom of one of the lens openings in the frame for a pair of glasses, or directly on the lens as shown in FIG. 4. In another embodiment, the source and sensor may be on separate mounts, so, for example, the source is on one side of the eye and the detector on the other. In another embodiment, the light is polarized and a second polarizing element, such as a polarizing filter is placed in front of the detector in order to provide sensitivity to a change in polarization of the reflected light. In another embodiment, the polarizing element is a polarizing beam splitter and two detectors are provided for the two orthogonal polarization directions in order to provide a difference signal that increases sensitivity to a change in polarization.

Note that a second sensor may also be mounted in front of the other eye to capture data from that eye as well, and more than one sensor may be used on each eye in order to monitor different areas of the eye. In one embodiment, the sensor is positioned so that both the source and detector view the lower portion of the eye adjacent to the pupil. In this manner, the source does not shine directly onto the retina and reflected light is collected from the portion of the eye that is at the most extreme portion of the blink, and thereby most strongly affected by incomplete blinking. In one embodiment, the source and detector are mounted in close proximity. In another embodiment, they are separate. For example, the source can mount above the eye and the detector below, in order to better capture direct reflection from the surface of the eye. In another example, the source is mounted on one side of the eye and the detector on the other (e.g., left and right side). In other embodiments, more than one source/detector combination is used to look at more than one location on the eye. For example, it is desirable to look at both the inner and outer extreme of the lower eye, for the purpose of seeing, for example, differences in tear film drying.

It is noted that the sclera is relatively featureless and white and the cornea is dark and featured. Thus, in one embodiment the light reflects from the sclera in order to provide an improved reflection signal and to reduce noise from eye movement or from reflection from fine features in the cornea. In another embodiment, light reflects from the cornea.

The source and sensor must be immune to environmental light and must operate outside visible light wavelengths (>640 nm). In one embodiment, the source emits at an infrared (>640 nm) wavelength such as 880 nm. In one embodiment, the detector has a bandpass filter that passes light of the source wavelength and blocks other wavelengths. In another embodiment, the source is modulated at a frequency such as 20 kHz, and the detector has an electronic filter that detects this modulation frequency, thereby rejecting other frequencies and dc light. An example circuit is shown in FIG. 5. The 880 nm source is driven with digital pulses from the processor. The detector is a phototransistor (TEMT1020) with peak sensitivity at 880 nm and an optical filter to reject other optical wavelengths. The phototransistor emitter voltage passes through a capacitor to remove the dc component, and then to a non-inverting op amp amplifier with 5× gain. In one embodiment the camera is sensitive to infrared light (>650 nm wavelength). An infrared LED is used to provide background illumination of the eye, and the camera has a cutoff filter that makes it insensitive to visible wavelengths.

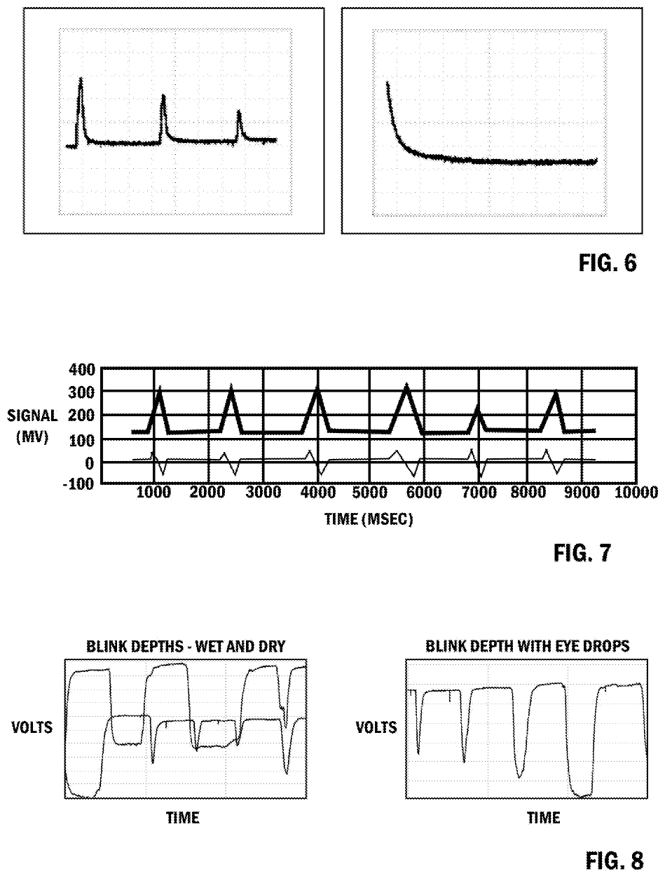

FIG. 6 shows signals from reduction to practice for this device. The left figure shows three blinks with progressively decreasing completeness. The right shows decay of the signal as the tear film evaporates.

In one mode of operation, the circuit turns on the source, reads the detector signal while the source is on, turns the source off, reads the signal a second time, and subtracts the off value from the on value. This removes noise, and allows use of a simple, compact circuit. The processor controls the timing. In one example, the source is turned on and the signal read 0.5 millisecond later. The source is then turned off and read 0.5 millisecond later. This provides a reading about once a millisecond, adequate to characterize blinks, which happen on a time scale of 20-100 milliseconds and at a time interval of a few seconds.

In one embodiment, the signal baseline is dynamically corrected. This is useful because, for example, the position of the eyeglass frame can change and affect the signal baseline. The dynamic correction can be implemented by methods such as measuring the signal between blinks and adjusting the baseline so the between blink signal is a constant value. Another method is to obtain an average signal for both between blinks and at the full depth of the blink, and to adjust the baseline and gain so these two values remain constant.

FIG. 3 illustrates how signals such as those shown in FIG. 4 provide information related to a blink. The signals are shown reversed in amplitude, so that a peak represents a minimum in the reflectance signal. The inverse of the time between peaks corresponds to the blink rate. The rise and fall times of the peak signals correspond to the rate of closure and opening of the eye. A peak of reduced amplitude corresponds to a partial or incomplete blink. The slope of the signal between peaks corresponds to a gradual drying of the eye. As long as the signal maintains a slope, the eye is still wet; if the signal stops changing, then the eye has fully dried in a time shorter than the time between blinks. Conversely, if the tear film is thick, the signal may not show any slope following a blink, as the drying time is long compared to the interval between blinks (see FIG. 8).

FIG. 7 shows blink signals recorded to a micro-SD memory card, and then post processed on a computer using Excel. Also shown is the derivative of the signal, which indicates the start and end of each blink to obtain blink rate and blink duration.

FIG. 8 shows reduction to practice in which an infrared LED and detector are mounted on the frame of a pair of eyeglasses, with the LED light shining on the lower outside portion of the sclera and the detector receiving light reflected from the sclera. The detected signal is converted to a 10-bit digital signal and sent at a rate of 400 points per sec (2.5 millisecond/point) via a Bluetooth® connection to a smart device such as an iPhone® or Android or an electronic watch. An app running on the smart device saves the data to a file for analysis and graphing.

The data samples in FIG. 8 show various features of the invention, including the ability to distinguish drying and incomplete vs. complete blinks. Note that without eye drops the signal rises after the blink as the thin tear film dries. When eye drops are added, the signal between blinks is flat because the thick tear film keeps the sclera moist between blinks. It is also possible to distinguish incomplete and complete blinks. The incomplete blinks have a sharp bottom and smaller amplitude, while the complete blinks have a flat bottom and a signal that indicates a gradual slowing of the eyelid near the bottom of the blink.

The blink monitoring device can be used as part of an ophthalmic exam. In one embodiment, the patient wears the device in the waiting room prior to or after the exam. In this manner blink data is acquired over an extended period without requiring the time of a clinician. In another embodiment, the blink monitoring device is used in conjunction with other diagnostic devices in the clinical setting. This makes it possible to record blink mechanics data in conjunction with a normal exam, saving time. For example, a patient can wear a blink monitor while undergoing a visual acuity exam. In another example, the patient can wear a blink monitor while sitting in an exam chair. In another example, the blink monitor is used in conjunction with a slit lamp. In each of these cases, the data can be sent directly to a clinician's computer so that it is available for analysis and guidance in treatment.

The device may be used to advise or guide treatment of conditions associated with blink mechanics. This may be based on information obtained while the patient wears the device in the clinic either before or during an exam, or while the patient wears the device while conducting activities outside the clinic.

The device may also be used as a method of treatment of conditions related to blink mechanics, either by itself or in conjunction with other interventions. In one example, it is found either through collecting data with the device or through observation, that the patient has irritation because of incomplete blinks. The device includes a feedback mechanism that indicates when the patient's blinking is incomplete. This could be one or more flash from an LED light, one or more sound, one or more vibration, or a combination of these. When the device detects incomplete blinking, it provides feedback, prompting the patient to blink completely. This provides training to improve the completeness of the patient's blinking. In one embodiment, the source of the feedback is the device mounted on the user's head. In another embodiment, the source of the feedback is a smart device the patient carries such as a smart phone or watch, which can provide any or all of several forms of feedback, such as sound, vibration, or a message on the screen in the form of text or a graphic. Similar feedback methods can be used for other aspects of blink mechanics, such as blink rate.

In another example, an exam determines that the patient can benefit from use of eye drops in order to maintain the tear film. The device determines when the tear film is drying too quickly, and prompts the patient to add eye drops. This enables the use of the eye drops exactly when needed.

In one embodiment, the patient wears the blink monitoring device during daily activity and the results are downloaded and read by a clinician. In one embodiment, the data is acquired on a handheld device such as a smart phone and automatically sent to the clinician's office for analysis through a phone or internet (e.g., Wi-Fi) connection.

The clinician may then use the information obtained from the device at least in part to recommend an approach to treatment. In one embodiment, the clinician uses information obtained on blink rate and tear film drying and, optionally, blink completeness, not just from the clinical setting, but also from routine activities. In one embodiment, the user has added notes that may relate to activities they were undertaking or how their eyes felt at a particular moment. The treatment may then employ a variety of methods such as medication, thermal stimulation, or wearing of the device to provide feedback and training to improve blink mechanics. The device may also provide a real-time indication of when to apply medication such as eye drops. The device may also be worn during or after a course of treatment to advice the clinician of the effectiveness of the treatment.

The invention can be used as part of treatment of a condition associated with abnormal blink mechanics, such as DES or Parkinson's Disease. In one embodiment it provides a feedback signal such as a flash or beep indicating when to blink. In another embodiment it provides a feedback signal such as a flash or beep indicating that blinks are incomplete. In another embodiment, it determines that the tear film is drying rapidly or substantially non-existent and indicates that the user should add eye drops.

The above description is intended to be illustrative, and not restrictive. For example, the above-described examples (or one or more aspects thereof) may be used in combination with each other. Other examples can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed example. Thus, the following claims are hereby incorporated into the Detailed Description as examples or examples, with each claim standing on its own as a separate example, and it is contemplated that such examples can be combined with each other in various combinations or permutations. The scope of the invention should be determined with reference to the appended claims, along with the full scope of equivalents to which such claims are entitled.

While the invention has been described in connection with what is presently considered to be the most practical and preferred embodiments, it is to be understood that the invention is not to be limited to the disclosed embodiments, but, on the contrary, is intended to cover various modifications and equivalent arrangements included within the spirit and scope of the appended claims.

I claim:

1. A system for monitoring eye blink mechanics, comprising:
a plurality of emitters;
a plurality of sensors;
a processor;
a data output; and
a battery;
wherein one of said plurality of sensors measures changes in reflectance of a surface on an eye based on a tear film content.

2. The invention of claim 1,
wherein one of said plurality of emitters comprises a light output having a peak wavelength longer than 640 nanometers.

3. The invention of claim 2,
wherein one of said plurality of sensors measures blink rate.

4. The invention of claim 2,
wherein one of said plurality of sensors measures tear film integrity.

5. The invention of claim 2,
wherein one of said plurality of sensors measures blink completeness.

6. The invention of claim 2,
wherein one of said plurality of sensors measures blink velocity.

7. The invention of claim 2, further comprising:
a wireless connectivity.

8. The invention of claim 2,
wherein said data output is to an external device carried by a user.

9. The invention of claim 2,
wherein one of said plurality of sensors measures changes in reflectance of said sclera of said eye based on tear film content.

10. The invention of claim 2, further comprising:
a light filter;
wherein said light filter selectively transmits light from said plurality of emitters.

11. The invention of claim 2,
an emission modulator;
wherein said emission modulator selectively modulates light intensity from said plurality of emitters.

12. The invention of claim 2,
wherein a first reflectance value reading is compared against a second reflectance value reading to calculate a change in reflectance.

13. A method for monitoring eye blink mechanics, comprising:
an eye blink mechanics monitor;
wherein said monitor provides a plurality of feedback from said eye blink mechanics monitor concerning a blink rate and a tear film drying rate.

14. The method of claim 13,
wherein one of said plurality of feedback comprises a light output;
wherein one of said plurality of feedback comprises a sound output; and
wherein one of said plurality of feedback comprises a tactile output.

15. The method of claim 13,
wherein said plurality of feedback is presented by a smart device.

16. The method of claim 13,
wherein said eye blink mechanics monitor measures blink completeness.

17. The method of claim 13,
wherein one of said plurality of feedback comprises an eye drops application indicator.

18. The method of claim 13,
wherein said plurality of feedback is used to inform creation of a treatment protocol for a blink-related pathology.

19. The method of claim 13,
wherein a plurality of measurements are obtained in a non-clinical setting.

20. The method of claim 13,
wherein said plurality of feedback is used to determine effectiveness of a treatment protocol.
